(19) United States
(12) Patent Application Publication
(10) Pub. No.: US 2026/0325498 A1
(43) Pub. Date: Oct. 22, 2026

(54) AN IMMEDIATE-RELEASE ORAL PHARMACEUTICAL FORM OF AMLODIPINE WITH INCREASED API CONTENT

(71) Applicant: Lasse KINAST, Berlin (DE)

(72) Inventor: Lasse KINAST, Berlin (DE)

(21) Appl. No.: 19/106,895

(22) PCT Filed: Sep. 7, 2023
(86) PCT No.: PCT/EP2023/074658

§ 371 Date: Feb. 26, 2025

(30) Foreign Application Priority Data
Sep. 7, 2022 (DE) 10 2022 003 339.9

(51) Int. Cl. A61K 31/4422; A61K 9/00; A61K 9/20

(52) U.S. Cl. CPC A61K 31/4422; A61K 9/0056; A61K 9/2009; A61K 9/2013; A61K 9/2018; A61K 9/2054; A61K 9/2059; A61K 9/2095

(57) ABSTRACT

The invention provides an immediate-release oral pharmaceutical composition which comprises by weight of 25.1% to 99.5% Amlodipine Besilate, at least 0.5% of disintegrant and from 0% to 74.4% additional pharmaceutical excipient.

The invention provides the composition of an immediate-release oral pharmaceutical form which comprises by weight of 25.1% to 99.5% Amlodipine Besilate, at least 0.5% of disintegrant and from 0% to 74.4% additional pharmaceutical excipient. With the composition invented, the prepared Amlodipine Besilate immediate-release oral pharmaceutical form has the benefit of a higher percentage Amlodipine Besilate content and a lowered percentage excipient content, while keeping with the requirements to the pharmaceutical quality.

### TECHNICAL FIELD

The invention belongs to the field of pharmaceutical preparations, a composition of matter, for the composition of an Amlodipine Besilate immediate-release oral pharmaceutical form.

### BACKGROUND

Hypertension, or high blood pressure, is a serious health issue in many countries. According to the National Heart Blood and Lung Institute, it is thought that about 1 in 3 adults in the United States alone have hypertension. Left unchecked, hypertension is considered a substantial risk factor for cardiovascular and other diseases including coronary heart disease, myocardial infarction, congestive heart failure, stroke, and kidney failure. Hypertension is classified as primary (essential) hypertension or secondary hypertension. Primary hypertension has no known cause and may be related to several environmental, lifestyle and genetic factors such as stress, obesity, smoking, inactivity, and sodium intake. Secondary hypertension can be caused by drug or surgical interventions or by abnormalities in the renal, cardiovascular, or endocrine system.

Amlodipine is a calcium channel blocker. It affects the movement of calcium into the cells of the heart and blood vessels. As a result, Amlodipine relaxes blood vessels and increases the supply of blood and oxygen to the heart while reducing its workload.

Amlodipine is currently administered in the form of oral tablets, (e.g., Norvasc) or in the form of a refrigerated liquid formulation. A typical tablet formulation of Amlodipine immediate-release oral pharmaceutical form contains around 2 to 25% Amlodipine Besilate. The standard dosages being from 2.5 mg to 10 mg Amlodipine, and the typical dosage form being tablet. Amlodipine is one of the most often prescribed medicines in the world. Amlodipine tablets are usually and out of necessity packaged in PVC/ALU or ALU/ALU blisters, to ensure the stability of the product.

The environmental burden of pharmaceutical packaging is immense. This is because the aluminium and plastics used to form the blister packaging are difficult and even impossible to recycle effectively as the materials are sealed together. Separating the materials again requires high amounts of energy and even then, the aluminium received from this is not of the same high quality as new aluminium. The larger the immediate-release oral pharmaceutical dosage form is, the larger the primary packaging must be and therefore also the more waste material is created.

Additionally, there is the environmental burden caused through the extensive transportation steps required of the finished product, such as the transportation from the manufacturer to the wholesaler and then to the pharmacy. Each box of finished product in the pharmaceutical industry needs to be transported several steps until it reaches the customer, often even global transportation routes are involved. This is especially the case for generic medicines, and further relevant for products which are sold in the millions, like with Amlodipine. The volume of the finished product determines how efficiently transportation and storage can be conducted, as a product with a high volume will require more space and more shipments as the same product at a smaller volume.

Furthermore, when it is avoidable to expose the patient to excipients, it should be done. An immediate-release oral pharmaceutical form is administered with the goal of subjecting the human body to the drug and not the excipients. It is a waste of resources of the pharma industry to use excipients in products where they can be avoided, especially when used in high quantities. Also, the global demand of excipients is ever increasing, and the supply capabilities of the chemical industry is falling behind the demand which makes this waste of recourses even more problematic.

Patented combinations of Amlodipine tablets, such as EP2359815A1 reach an Amlodipine content of 20.0% by weight, WO2004075825A2 reaches 2.5% by weight and EP1932528 manages to reach 2.0% to 10.0% by weight. The highest Amlodipine content of 25.0% has been found possible in the patent CN104739799B. In a 2004 Paper from Abdoh et al. (A. Abdoh et al., Amlodipine Besylate-Excipients Interaction in Solid Dosage Form; Pharmaceutical Development and Technology, 2004, Vol. 9, No. 1. pp. 15-24), the compatibility of amlodipine besylate in its solid formulations with various drug excipients was tested. Binary (1:1. w/w) mixtures or multicomponent mixtures (1:1:1, w/w) of amlodipine besilate and excipients were incubated under “stress stability” conditions (e.g. elevated temperature and/or relative humidity). However, the studies were focused on studying the stability of powders of amlodipine besylate and excipients under such conditions. Immediate-release oral pharmaceutical compositions were not prepared and the suitability of such mixtures for oral pharmaceutical compositions was therefore not apparent from this study. It is not evident from previous patents or studies that the Amlodipine content could be brought to any higher levels.

We have found a way to create an immediate-release oral pharmaceutical form of Amlodipine with an increased percentage amlodipine content that can help immensely with the listed problems. Currently, on the market Amlodipine 5 mg Tablets weigh roughly 120 mg, and Amlodipine 10 mg Tablets weigh roughly 190 mg. The excipient content is 113 mg per tablet and 176 mg per tablet respectively. Through this invention the excipient content can be brough down to 0.035 mg per 5 mg tablet and down to 0.070 mg per 10 mg tablet, making up to 99.96% of the excipient in the current tablets obsolete.

As the amount of excipient needed is reduced to a minimum, the immediate-release oral pharmaceutical form shrinks, which in turn shrinks the size of the blister and shrinks the size of the entire finished product. Now a previously impossibly small sized blister can be used, and this enables the finished product to have a fraction of the current volume. This in turn allows for much more efficient transportation, lowering the carbon footprint of the product, and only a fraction of the packaging waste is created.

It also reduces the amount of excipient administered to the patient down to the indispensable and drastically alleviates the pressure on the pharmaceutical industry to seek out and process an unnecessarily high quantity of excipient.

As Amlodipine is one of the most prescribed medicines in the world, it is synthesized in many production plants and the finished product is manufactured globally. The facilities and equipment needed to implement production of the given combination is readily available. The following examples of composition and preparation can realistically be realized at any production site intended for tabletting.

### BRIEF DESCRIPTION OF THE FIGURES

FIG. 1 shows a graph representing exemplary results of the dissolution study in the below examples.

### EXAMPLES

### Example 1—Example Preparation of Amlodipine Tablets

Step 1. All excipients of either Table 1 to 6 (the amounts being scaled according to the desired batch size), are passed through an appropriate screen. For the avoidance of doubt, a screen in this context refers to a sieve. Preferably sieves with a pore size of 250 μm-710 μm.
Step 2. All excipients other than any optional lubricant, are first blended for 5 minutes at 10 rpm in an appropriate blender.
   For the avoidance of doubt, in view of the below tables it is apparent that Amlodipine Besilate is added; it can be added in Step 1 or 2.
Step 3. Any optional lubricant is then added to the blend, and again blended for 3 minutes at 10 rpm.
Step 4. The final blend is compressed into tablets.
Step 5. A film coating can be added to the tablets.

### Example Compositions

TABLE 1
Composition Example 1

| Component | Composition (%) |
|---|---|
| Amlodipine Besilate | 99.5 |
| Sodium Starch Glycolate | 0.5 |
| Total | 100.0 |

TABLE 2
Composition Example 2

| Component | Composition (%) |
|---|---|
| Amlodipine Besilate | 40.8 |
| Sodium Starch Glycolate | 59.2 |
| Total | 100 |

TABLE 3
Composition Example 3

| Component | Composition (%) |
|---|---|
| Amlodipine Besilate | 40.8 |
| Sodium Starch Glycolate | 58.2 |
| Magnesium Stearate | 1.0 |
| Total | 100 |

TABLE 4

| Composition Example 4 | |
|---|---|
| Component | Composition (%) |
| Amlodipine Besilate | 40.8 |
| Sodium Starch Glycolate | 1.0 |
| Microcrystalline Cellulose | 57.2 |
| Magnesium Stearate | 1.0 |
| Total | 100 |

TABLE 5

| Composition Example 5 | |
|---|---|
| Component | Composition (%) |
| Amlodipine Besilate | 40.8 |
| Sodium Starch Glycolate | 0.5 |
| Microcrystalline Cellulose | 56.7 |
| Silicon Dioxide | 1.0 |
| Magnesium Stearate | 1.0 |
| Total | 100 |

TABLE 6

| Composition Example 6 | |
|---|---|
| Component | Composition (%) |
| Amlodipine Besilate | 27.5 |
| Sodium Starch Glycolate | 0.5 |
| Microcrystalline Cellulose | 70.0 |
| Silicon Dioxide | 1.0 |
| Magnesium Stearate | 1.0 |
| Total | 100 |

TABLE 7

| Composition Example 7 | |
|---|---|
| Component | Composition (%) |
| Amlodipine Besilate | 99.5 |
| Croscarmellose Sodium | 0.5 |
| Total | 100.0 |

TABLE 8

| Composition Example 8 | |
|---|---|
| Component | Composition (%) |
| Amlodipine Besilate | 99.5 |
| Microcrystalline Cellulose | 0.5 |
| Total | 100.0 |

TABLE 9

| Composition Example 9 | |
|---|---|
| Component | Composition (%) |
| Amlodipine Besilate | 99.5 |
| Crospovidone | 0.5 |
| Total | 100.0 |

TABLE 10
Composition Example 10

| Component | Composition (%) |
|---|---|
| Amlodipine Besilate | 99.5 |
| Croscarmellose Sodium | 0.25 |
| Sodium Starch Glycolate | 0.25 |
| Total | 100.0 |

TABLE 11
Composition Example 11

| Component | Composition (%) |
|---|---|
| Amlodipine Besilate | 27.5 |
| Croscarmellose Sodium | 70.5 |
| Silicon Dioxide | 1.0 |
| Sodium Lauryl Sulphate | 1.0 |
| Total | 100.0 |

TABLE 12
Composition Example 12

| Component | Composition (%) |
|---|---|
| Amlodipine Besilate | 27.5 |
| Sodium Starch Glycolate | 1.5 |
| Lactose | 70.0 |
| Magnesium Stearate | 1.0 |
| Total | 100.0 |

TABLE 13

| Composition Example 13 | |
|---|---|
| Component | Composition (%) |
| Amlodipine Besilate | 27.5 |
| Starch | 1.5 |
| Microcrystalline Cellulose | 70.0 |
| Talc | 1.0 |
| Total | 100.0 |

TABLE 14
Composition Example 14

| Component | Composition (%) |
|---|---|
| Amlodipine Besilate | 81.5 |
| Sodium Starch Glycolate | 0.5 |
| Lactose | 15.0 |
| Sodium Lauryl Sulphate | 3.0 |
| Total | 100.0 |

### Example 2—Example Tablet Weights Form Different Compositions

TABLE 15
Overview of example tablet weights

| Composition Example # | Example Dosage | Example Total Weight |
|---|---|---|
| Composition Example 1 | 10 mg Tablet | 13.94 mg |
| Composition Example 1 | 5 mg Tablet | 6.97 mg |
| Composition Example 2 | 10 mg Tablet | 34.00 mg |
| Composition Example 3 | 5 mg Tablet | 17.00 mg |
| Composition Example 6 | 10 mg Tablet | 50.44 mg |
| Composition Example 7 | 10 mg Tablet | 13.94 mg |
| Composition Example 8 | 10 mg Tablet | 13.94 mg |
| Composition Example 9 | 10 mg Tablet | 13.94 mg |
| Composition Example 11 | 10 mg Tablet | 50.44 mg |
| Composition Example 12 | 5 mg Tablet | 25.22 mg |
| Composition Example 14 | 10 mg Tablet | 17.02 mg |

### Example 3—Analytical Results

TABLE 16
Disintegration Time & Resistance to Crushing

| Example | Disintegration Time | Resistance to Crushing |
|---|---|---|
| Composition | (Minutes) | (N) |
| 1 | <2 min | ≈20N |
| 2 | <2 min | ≈20N |
| 3 | <2 min | ≈20N |
| 4 | <2 min | ≈20N |
| 5 | <2 min | ≈20N |
| 6 | <2 min | ≈20N |
| 7 | <2 min | ≈20N |
| 8 | <2 min | ≈15N |
| 9 | <2 min | ≈20N |
| 10 | <2 min | ≈20N |
| 11 | <2 min | ≈20N |
| 12 | <2 min | ≈25N |
| 13 | <2 min | ≈25N |
| 14 | <2 min | ≈20N |

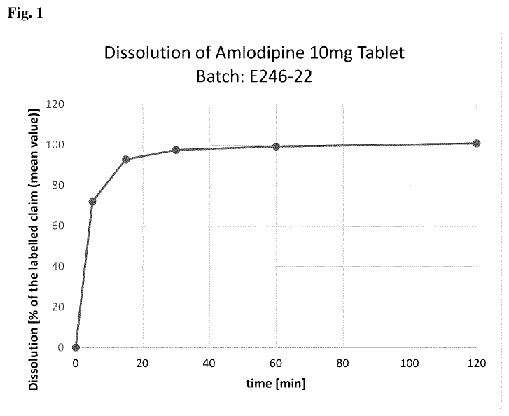

A dissolution study was conducted on a tablet of Example 3 in 900 ml of pH 6.8 buffer at 37° C., USP Apparatus 1 set to 100 rpm. The resultant dissolution curve demonstrates that the tablet rapidly dissolved with complete dissolution within 30 minutes. The results are shown in Table 7 and FIG. 1.

TABLE 17
Data of the dissolution study in tabulated form

| time [min] | 0 | 5 | 15 | 30 | 60 | 120 |
|---|---|---|---|---|---|---|
| Tablet 1 (mean of two injections) | 0 | 74.7 | 92.0 | 98.8 | 100.2 | 99.7 |
| Tablet 2 (mean of two injections) | 0 | 70.9 | 91.8 | 96.3 | 99.4 | 101.4 |
| Tablet 3 (mean of two injections) | 0 | 69.8 | 94.8 | 95.9 | 99.2 | 100.6 |
| Tablet 4 (mean of two injections) | 0 | 70.1 | 92.9 | 98.3 | 97.3 | 100.3 |
| Tablet 5 (mean of two injections) | 0 | 74.8 | 91.0 | 97.3 | 99.3 | 102.3 |
| Tablet 6 (mean of two injections) | 0 | 71.3 | 94.3 | 97.6 | 99.3 | 99.8 |
| min value [%] | 0 | 69.8 | 91.0 | 95.9 | 97.3 | 99.7 |
| max value [%] | 0 | 74.8 | 94.8 | 98.8 | 100.2 | 102.3 |
| confidence area | 0 | 2.0 | 1.3 | 1.0 | 0.9 | 0.9 |
| Dissolution [% of the labelled | 0 | 71.9 | 92.8 | 97.4 | 99.1 | 100.7 |
| claim (mean value)] | | | | | | |

The suitability of the quality of the immediate-release oral pharmaceutical form made under the invention is met, as it fulfills the requirements of the USP monograph for Amlodipine Tablets.

Certain embodiments of the invention are enumerated in the following:
1. A pharmaceutical composition of an immediate-release oral pharmaceutical form which comprises by weight of 25.1% to 99.5% Amlodipine Besilate, at least 0.5% of disintegrant and from 0% to 74.4% additional pharmaceutical excipient. The preferred amount of Amlodipine Besilate being 30.1% to 81.5% by weight.
2. The pharmaceutical excipient of embodiment 1 consists of, but is not limited to, one or more disintegrant, lubricant, filler and glidant.
3. The disintegrant of embodiment 1 and embodiment 2 consists of, but is not limited to microcrystalline cellulose, sodium alginate, sodium starch glycolate, croscarmellose sodium, crospovidone and any “super-disintegrants”.
4. The filler of embodiment 2 consists of, but is not limited to cellulose, lactose, mannitol, starch, sucrose, and calcium phosphate.
5. The lubricant of embodiment 2 consists of, but is not limited to calcium stearate, magnesium stearate, stearic acid, sodium lauryl sulphate, and magnesium lauryl sulphate.
6. The glidant of embodiment 2 consists of, but is not limited to silica derivatives, talc, and corn starch.
7. The immediate-release oral pharmaceutical form of embodiment 1 entails the tablet form or the film-coated tablet form. In the case of film-coated tablet form, the percentage weight refers to the weight of the tablet core.
8. The Amlodipine Besilate of embodiment 1 entails also the other pharmaceutically acceptable salts of Amlodipine.
9. The composition of embodiment 1 is for the dosage range of 2.5 mg Amlodipine to 20 mg Amlodipine. The preferred dosage range is from of 5 mg Amlodipine to 10 mg Amlodipine.

Further embodiments of the invention are enumerated in the following:
1. An immediate-release oral pharmaceutical composition comprising by weight of 25.1% to 99.5% Amlodipine Besilate, at least 0.5% of disintegrant and from 0% to 74.4% additional pharmaceutical excipient.
2. Immediate-release oral pharmaceutical composition according to embodiment 1, wherein the amount of Amlodipine Besilate is 30.1% to 81.5% by weight.
   In another embodiment, the amount of Amlodipine Besilate in the immediate-release oral pharmaceutical composition is more than 50% to 99.5% by weight, preferably 75% to 99.5% by weight, more preferably 80% to 99.5% by weight, more preferably 81.5% to 99.5% by weight.
3. The immediate-release oral pharmaceutical composition according to embodiment 1 or 2, wherein the additional pharmaceutical excipient comprises one or more components selected from the group comprising disintegrant, lubricant, filler and glidant.
4. The immediate-release oral pharmaceutical composition according to any one of embodiments 1 to 3, wherein the disintegrant is one or more disintegrants selected from the group comprising microcrystalline cellulose, sodium croscarmellose, carboxymethyl cellulose, alginic acid, sodium alginate and their derivatives, sodium starch glycolate (such as Glycolys®, Explotab®, Vivastar® P), croscarmellose sodium (such as Ac Di Sol®, Solutab®, Vivasol®), crospovidone (such as Kollidon®, Kollicoat®) and other super-disintegrants, as well as cellulose, lactose, mannitol, starch and sucrose.
   Super disintegrants are disintegrants that typically can be used in small concentrations, such as 0.5 to 5% in typical pharmaceutical formulations, while still achieving suitable disintegrant effect. Examples of other super-disintegrants according the present invention are disintegrants, such as L-hydroxy propyl cellulose, Sodium carboxymethyl starch (such as Primogel®), magnesium aluminum silicate (such as Veegum® HV), calcium silicate, cross-linked alginic acid (also known as alginic acid National Formulary (NF), such as Satialgine®), Soy derivatives/polysaccharides (such as Emcosoy®).
5. The immediate-release oral pharmaceutical composition according to any one of embodiment 3 or 4, wherein the filler is one or more fillers selected from the group comprising cellulose, lactose, mannitol, starch, sucrose, and calcium phosphate, calcium carbonate, maltodextrin, sorbitol and dextrin.
   It is known to the skilled person that certain excipients used as fillers can also act as disintegrant, such as cellulose, lactose, mannitol, starch and sucrose. In embodiments where one of these is included as disintegrant, they are not additionally used as fillers, in other words, their inclusion as “disintegrant” or “filler” in the embodiments of the present invention is mutually exclusive.
6. The immediate-release oral pharmaceutical composition according to any one of embodiments 3 to 5, wherein the lubricant is one or more lubricants selected from the group comprising calcium stearate, magnesium stearate, stearic acid, sodium lauryl sulphate, and magnesium lauryl sulphate.
7. The immediate-release oral pharmaceutical composition according to any one of embodiments 3 to 6, wherein the glidant is one or more glidants selected from the group comprising silica derivatives, such as silicone dioxide, talc, and corn starch.
8. The immediate-release oral pharmaceutical composition of any one of embodiments 1 to 7, which is in tablet form or in a film-coated tablet form, wherein in said film-coated tablet form the percentage weight refers to the weight of the tablet core.
9. The immediate-release oral pharmaceutical composition of any one of embodiments 1 to 8, wherein Amlodipine Besilate is comprised in a dosage range of 2.5 mg to 20 mg, preferably 5 mg to 10 mg.
10. A method of preparing a tablet comprising a composition according to any one of embodiments 1 to 9, the method comprising
   a) passing comprises by weight of 25.1% to 99.5% Amlodipine Besilate, at least 0.5% of disintegrant and from 0% to 74.4% additional pharmaceutical excipient through a screen,
   b) blending all excipients other than lubricant,
   c) optionally adding lubricant to the blend of c), and blend,
   d) compressing the blend obtained from c) into tablets, and
   e) optionally adding a film coating to the tablets, wherein Amlodipine Besilate is added either in step a) or b).

1. An immediate-release oral pharmaceutical composition comprising by weight of 25.1% to 99.5% amlodipine besylate, at least 0.5% of disintegrant and from 0% to 74.4% additional pharmaceutical excipient.

2. The immediate-release oral pharmaceutical composition according to claim 1, wherein the amount of amlodipine besylate is 30.1% to 81.5% by weight.

3. The immediate-release oral pharmaceutical composition according to claim 1, wherein the additional pharmaceutical excipient comprises one or more components selected from disintegrant, lubricant, filler, and glidant.

4. The immediate-release oral pharmaceutical composition according to claim 1, wherein the disintegrant is one or more disintegrants selected from microcrystalline cellulose, sodium croscarmellose, carboxymethyl cellulose, alginic acid, sodium alginate, and their derivatives, sodium starch glycolate (such as Glycolys®, Explotab®, Vivastar® P), croscarmellose sodium (such as Ac Di Sol®, Solutab®, Vivasol®), crospovidone (such as Kollidon®, Kollicoat®) and other super-disintegrants, as well as cellulose, lactose, mannitol, starch, and sucrose.

5. The immediate-release oral pharmaceutical composition according to claim 3, wherein the filler is one or more fillers selected from cellulose, lactose, mannitol, starch, sucrose, calcium phosphate, calcium carbonate, maltodextrin, sorbitol, and dextrin.

6. The immediate-release oral pharmaceutical composition according to claim 3, wherein the lubricant is one or more lubricants selected from the group comprising calcium stearate, magnesium stearate, stearic acid, sodium lauryl sulphate, and magnesium lauryl sulphate.

7. The immediate-release oral pharmaceutical composition according to claim 3, wherein the glidant is one or more glidants selected from silica derivatives, such as silicone dioxide, talc, and corn starch.

8. The immediate-release oral pharmaceutical composition according to claim 1, wherein the composition is in tablet form or in a film-coated tablet form, wherein in said film-coated tablet form the percentage weight refers to the weight of the tablet core.

9. The immediate-release oral pharmaceutical composition according to claim 1, wherein amlodipine besylate is comprised in a dosage range of 2.5 mg to 20 mg.

10. A method of preparing a tablet comprising a composition according to claim 1, the method comprising:
a) passing comprises by weight of 25.1% to 99.5% amlodipine besylate, at least 0.5% of disintegrant, and from 0% to 74.4% additional pharmaceutical excipient through a screen,
b) blending all excipients other than lubricant,
c) optionally adding lubricant to the blend of c), and blend,
d) compressing the blend obtained from c) into tablets, and
e) optionally adding a film coating to the tablets,
wherein amlodipine besylate is added either in step a) or b).

11. The immediate-release oral pharmaceutical composition according to claim 1, wherein amlodipine besylate is comprised in a dosage range of 5 mg to 10 mg.

12. The immediate-release oral pharmaceutical composition according to claim 1, wherein the amount of amlodipine besylate is 75% to 99.5% by weight.

13. The immediate-release oral pharmaceutical composition according to claim 1, wherein the amount of amlodipine besylate is 80% to 99.5% by weight.

14. The immediate-release oral pharmaceutical composition according to claim 1, wherein the amount of amlodipine besylate is 81.5% to 99.5% by weight.

15. The immediate-release oral pharmaceutical composition according to claim 1, wherein the amount of amlodipine besylate is 27.5% to 99.5% by weight.
